(19) United States
(12) Patent Application Publication
(10) Pub. No.: US 2023/0372461 A1
(43) Pub. Date: Nov. 23, 2023

(54) IMMUNOGENIC PEPTIDES, COMPOSITIONS, AND METHODS FOR THE TREATMENT AND/OR PREVENTION OF MALARIA

(71) Applicants: THE HOSPITAL FOR SICK CHILDREN, Toronto (CA); Deutsches Krebsforschungszentrum Stiftung des öffentlichen Rechts, Heidelberg (DE); MAX-PLANCK-GESELLSCHAFT ZUR FOERDERUNG DER WISSENSCHAFTEN E.V., Berlin (DE)

(72) Inventors: Jean-Philippe Julien, Toronto (CA); Stephen Scally, Toronto (CA); Katherine Prieto, Toronto (CA); Hedda Wardemann, Heidelberg (DE); Julia Ludwig, Heidelberg (DE); Rajagopal Murugan, Heidelberg (DE); Giulia Costa, Berlin (DE); Elena Levashina, Berlin (DE)

(21) Appl. No.: 17/767,476

(22) PCT Filed: Oct. 9, 2020
(86) PCT No.: PCT/CA2020/051360

§ 371 Date: Apr. 8, 2022

Related U.S. Application Data
(60) Provisional application No. 62/912,972, filed on Oct. 9, 2019.

(51) Int. Cl. A61K 39/015; A61K 39/385; A61P 37/04; C07K 14/445

(52) U.S. Cl. CPC A61K 39/015; A61K 39/385; A61P 37/04; C07K 14/445; A61K 2039/55555

(57) ABSTRACT

Described herein is an immunogenic fusion protein comprising: an immunogenic peptide or an immunogenic variant thereof, the immunogenic peptide comprising the following motifs: —KQPAₐ; QPAKₐ; PAKQₐ; or AKQPₐ; —NPDP_{b}; PNPD_{b}; DPNP_{b}; or PNPD_{b}; —NANP_{c}; ANPN_{c}; NPNA_{c}; or PNAN_{c}; —NVDP_{d}; VDPN_{d}; DPNV_{d}; or PNVD_{d}; and —NANPₑ; ANPNₑ; NPNAₑ; or PNANₑ. wherein a, b, c, d, and e are each independently 0 or greater and wherein a±b±c±d±e is at least 2; and a nanocage monomer peptide.

### FIELD

The present invention relates to fusion proteins. In particular, the present invention relates to fusion proteins, vaccines comprising the fusion proteins, and related compositions and methods.

### BACKGROUND

Plasmodium falciparum (Pf) is the parasite that accounts for most malaria fatalities globally, but a highly efficacious pre-erythrocytic subunit vaccine remains elusive. Previous studies, such as Foquet, L. et al. (J. Clin. Invest. 124, 140-144, 2014), White, M. T. et al. (PLoS One 8, e61395, 2013), RTSS Clinical Trials Partnership (Lancet 386, 31-45, 2015), and Sumitani, M. et al. (Insect Mol. Biol. 22, 41-51, 2013), have shown that protective antibodies against malaria exist that recognize the NANP repeat of the CSP protein on the surface of the parasite.

The RTS,S/AS01 malaria subunit vaccine (GSK) contains 18.5 CSP NANP-NVDP repeats and the complete C-CSP domain, displayed on a virus-like particle composed of Hepatitis B surface antigen building blocks. RTS,S/AS01 protected approximately 50% of vaccinated individuals in a recent phase Ill trial in Africa, but its efficacy waned rapidly (Agnandji et al., 2011; RTSS Clinical Trials Partnership, 2015).

U.S. Patent Application Publication Nos. 2013/0259890 and 2016/0038580 describe a nucleotide sequence and other constructs used for expression of recombinant P. falciparum circumsporozoite proteins in bacterial cells such as E. coli. Processes for producing a soluble recombinant P. falciparum CSP from E. coli are described. Methods to produce a human-grade, highly immunogenic anti-malaria vaccine based on CSP are shown. The recombinant P. falciparum circumsporozoite protein by itself or in combination with other malaria antigens or adjuvants are described as forming the basis of an effective malaria vaccine.

International Patent Application Publication No. 2018/193063 describes a fragment of Plasmodium circumsporozoite protein, for example for use in a malaria vaccine, nucleic acids encoding a fragment of Plasmodium circumsporozoite protein, compositions comprising a fragment of Plasmodium circumsporozoite protein, and antibodies binding to a fragment of Plasmodium circumsporozoite protein. The antibodies bind specifically to P. falciparum sporozoites and may be used in the treatment and/or prevention of malaria.

International Patent Application Publication No. 2012/154199 describes nucleotide sequences and other constructs used for expression of recombinant P. falciparum circumsporozoite proteins in bacterial cells such as E. coli. Processes are described for producing a soluble recombinant P. falciparum CSP from E. coli. Methods to produce a human-grade, highly immunogenic anti-malaria vaccine based on CSP are described.

A need exists for the development of an effective malaria vaccine as well as alternative vaccine platforms and related vaccines, compositions, and methods.

### SUMMARY

In accordance with an aspect, there is provided an immunogenic fusion protein comprising:
an immunogenic peptide or an immunogenic variant thereof, the immunogenic peptide comprising the following motifs:
   KQPAₐ; QPAKₐ; PAKQₐ; or AKQPₐ;
   NPDP_{b}; PDPN_{b}; DPNP_{b}; or PNPD_{b};
   NANP_{c}; ANPN_{c}; NPNA_{c}; or PNAN_{c};
   NVDP_{d}; VDPN_{d}; DPNV_{d}; or PNVD_{d}; and
   NANPₑ; ANPNₑ; NPNAₑ; or PNANₑ.
   wherein a, b, c, d, and e are each independently 0 or greater and wherein a+b+c+d+e is at least 2; and
a nanocage monomer peptide.

In an aspect, a, b, c, d, e, or any combination thereof are each independently at least about 1.

In an aspect, a, b, c, d, e, or any combination thereof are each independently from about 1 to about 40.

In an aspect, a, b, c, d, and e are each independently from about 1 to about 100, such as from about 1, about 2, about 3, about 4, about 5, about 6, about 7, about 8, about 9, about 10, about 11, about 12, about 13, about 14, about 15, about 16, about 17, about 18, about 19, or about 20 to about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 60, about 70, about 75, about 80, about 90, or about 100, such as from about 1 to about 40, or about 1 to about 20, or from 1 to about 10.

In an aspect, a is 1.

In an aspect, b is 1.

In an aspect, c is 1.

In an aspect, d is 3.

In an aspect, e is 5 or 5.5.

In an aspect, e is 18.5.

In an aspect, when a, b, c, d, and/or e are greater than 1 such that the respective motif is at least partially repeated, the repeated motifs are each independently contiguous.

In an aspect, when a, b, c, d, and/or e are greater than 1 such that the respective motif is at least partially repeated, the repeated motifs are each independently non-contiguous.

In an aspect, the motifs are in the order KQPAₐ-NPDP_{b}-NANP_{c}-NVDP_{d}-NANPₑ.

In an aspect, the variant comprises at least about 70%, 75%, 80%, 85%, 90%, 95%, 97%, or 99% sequence identity to the immunogenic peptide.

In an aspect, the nanocage monomer is ferritin, apoferritin, encapsulin, SOR, lumazine synthase, pyruvate dehydrogenase, carboxysome, vault proteins, GroEL, heat shock protein, E2P, or MS2 coat protein, or fragments thereof, or variants thereof.

In an aspect, the nanocage monomer is provided as two or more self-assembling subunits.

In an aspect, the nanocage monomer peptide is from Helicobacter pylori.

In an aspect, the nanocage monomer peptide is not human.

In an aspect, the nanocage monomer comprises the amino acid sequence:

MLSKDIIKLLNEQVNKEMNSSNLYMSMSSWCYTHSLDGAGLFLFDHA
AEEYEHAKKLIVFLNENNVPVQLTSISAPEHKFEGLTQIFQKAYEHE
QHISESINNIVDHAIKSKDHATFNFLQWYVAEQHEEEVLFKDILDKI
ELIGNENHGLYLADQYVKGIAKSRKS

or a functional variant having at least 70% sequence identity thereto, or a functional fragment of either thereof.

In an aspect, the nanocage monomer peptide is modified to reduce an anti-nanocage monomer peptide immune response.

In an aspect, the nanocage monomer peptide is at least partially or fully masked.

In an aspect, the nanocage monomer peptide is at least partially glycan masked.

In an aspect, the nanocage monomer peptide is fully glycan masked.

In an aspect, the nanocage monomer comprises at least one NXT and/or NXS glycosylation motif.

In an aspect, the nanocage monomer comprises the amino acid sequence:

MLSKDIIKLLNEQVNKEMNSSNLYMSMSSWCYTHSLDGAGLFLFDHAAE
EYEHAKKLIVFLNENNVPVQLTSISAPEHKFEGLTQIFQKAYEHEQHIS
ESINNIVDHAIKSKDHATFNFLQWYVAEQHEEEVLFKDILDKIELIGNE
NHGLYLADQYVKGIAKSRKS

or a functional variant having at least 70% sequence identity thereto, or a functional fragment of either thereof, and wherein the sequence comprises a K77N and E79T mutation and/or an E99N and I101T mutations.

In an aspect, a plurality of the nanocage monomer peptides self-assemble into a nanocage.

In an aspect, the immunogenic peptide decorates the interior and/or exterior surface of the nanocage.

In an aspect, the fusion protein further comprises a peptide that provides exogenous T cell help and/or a peptide that provides autologous T cell help.

In an aspect, the peptide that provides exogenous T cell help comprises a PADRE peptide and/or a peptide derived from a pathogenic molecule, such as a tetanus toxoid peptide.

In an aspect, the PADRE peptide comprises the amino acid sequence AKFVAAWTLKAAA, or a functional variant thereof having at least 70% sequence identity thereto or a fragment of either thereof.

In an aspect, the peptide that provides autologous T cell help comprises a PfCSP T cell peptide epitope.

In an aspect, the peptide that provides exogenous T cell help and/or the peptide that provides autologous T cell help independently decorates the interior and/or exterior surface of the assembled nanocage.

In an aspect, the fusion protein further comprises a linker between any one or more of the motifs, the nanocage monomer, and any further peptides, such as the peptide that provides exogenous T cell help and/or the peptide that provide autologous T cell help.

In an aspect, the linker is a GGS linker.

In an aspect, the linker comprises the amino acid sequence:

GGS;
GGGGSGGSGGSGGS;
and/or
GGGGGSGGSGGSGGS.

In an aspect, the fusion protein comprises or consists of the sequence:
KQPA-NPDP-NANPNVDP3-NANP5-Hpferr-PADRE;
KQPA-NPDP-NANPNVDP3-NANP18.5-Hpferr-PADRE;
KQPA-NPDP-NANPNVDP3-NANP5-LS-PADRE; and/or
KQPA-NPDP-NANPNVDP3-NANP18.5-LS-PADRE.

MGILPSPGMPALLSLVSLLSVLLMGCVAETGWSHPQFEKLKENLYFQGKQPADGNPDPNAN
PNVDPNANPNVDPNANPNVDPNANPNANPNANPNANPNANPSRGGGGGSGGSGGSGGSDIIKLLN
EQVNKEMQSSNLYMSMSSWCYTHSLDGAGLFLFDHAAEEYEHAKKLIIFLNENNVPVQLTSISAPEHK
FEGLTQIFQKAYEHEQHISESINNIVDHAIKSKDHATFNFLQWYVAEQHEEEVLFKDILDKIELIGNENH
GLYLADQYVKGIAKSRKSGGSASAKFVAAWTLKAAA;
MGILPSPGMPALLSLVSLLSVLLMGCVAETGWSHPQFEKLKENLYFQGKQPADGNPDPNAN
PNVDPNANPNVDPNANPNVDPNANPNANPNANPNANPNANPNANPNANPNANPNANPNANPNANP
NANPNANPNANPNANPNANPNANPNANPSRGGGGGSGGSGGSGGSDIIKLLNEQVNKEMQSSNLY
MSMSSWCYTHSLDGAGLFLFDHAAEEYEHAKKLIIFLNENNVPVQLTSISAPEHKFEGLTQIFQKAYEH
EQHISESINNIVDHAIKSKDHATFNFLQWYVAEQHEEEVLFKDILDKIELIGNENHGLYLADQYVKGIAK
SRKSGGSASAKFVAAWTLKAAA;
MGILPSPGMPALLSLVSLLSVLLMGCVAETGWSHPQFEKLKENLYFQGKQPADGNPDPNAN
PNVDPNANPNVDPNANPNVDPNANPNANPNANPNANPNANPSRGGGGGSGGSGGSGGSMQIYEG
KLTAEGLRFGIVASRFNHALVDRLVEGAIDAIVRHGGREEDITLVRVPGSWEIPVAAGELARKEDIDAVI
AIGVLIRGATPHFDYIASEVSKGLADLSLELRKPITFGVITADTLEQAIERAGTKHGNKGWEAALSAIEM
ANLFKSLRGGSASAKFVAAWTLKAAA;
MGILPSPGMPALLSLVSLLSVLLMGCVAETGWSHPQFEKLKENLYFQGKQPADGNPDPNAN
PNVDPNANPNVDPNANPNVDPNANPNANPNANPNANPNANPNANPNANPNANPNANPNANPNANP
NANPNANPNANPNANPNANPNANPNANPSRGGGGGSGGSGGSGGSMQIYEGKLTAEGLRFGIVAS
RFNHALVDRLVEGAIDAIVRHGGREEDITLVRVPGSWEIPVAAGELARKEDIDAVIAIGVLIRGATPHFD
YIASEVSKGLADLSLELRKPITFGVITADTLEQAIERAGTKHGNKGWEAALSAIEMANLFKSLRGGSAS
AKFVAAWTLKAAA;
and/or
MGILPSPGMPALLSLVSLLSVLLMGCVAETGWSHPQFEKLKENLYFQGKQPADGNPDPNAN
PNVDPNANPNVDPNANPNVDPNANPNANPNANPNANPNANPNANPNANPNANPNANPNANPNANP
NANPNANPNANPNANPNANPNANPNANPSRGGGGGSGGSGGSGGSDIIKLLNEQVNKEMQSSNLY
MSMSSWCYTHSLDGAGLFLFDHAAEEYEHAKKLIIFLNENNVPVQLTSISAPEHNFTGLTQIFQKAYEH
EQHISNSTNNIVDHAIKSKDHATFNFLQWYVAEQHEEEVLFKDILDKIELIGNENHGLYLADQYVKGIAK
SRKSGGSASAKFVAAWTLKAAA;

or functional variants or fragments thereof.

In accordance with an aspect, there is provided a nucleic acid molecule encoding the fusion protein described herein.

In accordance with an aspect, there is provided a vector comprising the nucleic acid molecule described herein.

In accordance with an aspect, there is provided a host cell comprising the vector described herein and producing the fusion protein described herein.

In accordance with an aspect, there is provided a vaccine comprising the fusion protein described herein.

In an aspect, the vaccine further comprises an adjuvant.

In accordance with an aspect, there is provided an antibody that binds to the fusion protein described herein.

In accordance with an aspect, there is provided a highly efficacious pre-erythrocytic subunit malaria vaccine.

In accordance with an aspect, there is provided a C-terminal truncated PfCSP antigen.

In accordance with an aspect, there is provided a boostable malaria vaccine.

In an aspect, the vaccine is PfCSP-based.

In accordance with an aspect, there is provided an anti-malaria immunogenic peptide comprising KQPAₐ, wherein a is at least about 1.

In accordance with an aspect, there is provided a glycan-masked nanocage monomer peptide.

In an aspect, the nanocage monomer is ferritin, apoferritin, encapsulin, SOR, lumazine synthase, pyruvate dehydrogenase, carboxysome, vault proteins, GroEL, heat shock protein, E2P, or MS2 coat protein, or fragments thereof, or variants thereof.

In an aspect, the nanocage monomer is provided as two or more self-assembling subunits.

In an aspect, the nanocage monomer peptide is from Helicobacter pylori.

In an aspect, the nanocage monomer peptide is not human.

In an aspect, the peptide further comprises a bioactive moiety.

In an aspect, the bioactive moiety comprises an antibody or fragment thereof, an antigen, a detectable moiety, a pharmaceutical agent, a diagnostic agent, or combinations thereof.

In an aspect, the bioactive moiety comprises an antigen.

In an aspect, a plurality of the nanocage monomer peptides self-assemble into a nanocage.

In an aspect, the bioactive moiety decorates the interior and/or exterior surface of the nanocage.

In an aspect, the nanocage monomer peptide is at least partially or fully masked.

In an aspect, the nanocage monomer peptide is at least partially glycan masked.

In an aspect, the nanocage monomer peptide is fully glycan masked.

In an aspect, the nanocage monomer comprises at least one NXT and/or NXS glycosylation motif.

In an aspect, the nanocage monomer comprises the amino acid sequence:

MLSKDIIKLLNEQVNKEMNSSNLYMSMSSWCYTHSLDGAGLFLFDHAAE
EYEHAKKLIVFLNENNVPVQLTSISAPEHKFEGLTQIFQKAYEHEQHIS
ESINNIVDHAIKSKDHATFNFLQWYVAEQHEEEVLFKDILDKIELIGNE
NHGLYLADQYVKGIAKSRKS

or a functional variant having at least 70% sequence identity thereto, or a functional fragment of either thereof, and wherein the sequence comprises a K77N and E79T mutation and/or an E99N and I101T mutations.

In accordance with an aspect, there is provided a method of treating and/or preventing malaria, comprising administering the protein, peptide, nucleotide, vector, or cell described herein.

In accordance with an aspect, there is provided a use of the protein, peptide, nucleotide, vector, or cell described herein for treating and/or preventing malaria.

In accordance with an aspect, there is provided a protein, peptide, nucleotide, vector, or cell as described herein for treating and/or preventing malaria.

In an aspect, the preventative and/or treatment effect is boostable.

In an aspect, the preventative and/or treatment effect persists for at least about 6 months or more, such as about 9 months or more, about 12 months or more, about 1, about 2, about 3, about 4, about 5, about 6, about 7, about 8, about 9, or about 10 years or more.

The novel features of the present invention will become apparent to those of skill in the art upon examination of the following detailed description of the invention. It should be understood, however, that the detailed description of the invention and the specific examples presented, while indicating certain aspects of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The present invention will be further understood from the following description with reference to the Figures, in which:

FIG. 1 shows the PfCSP sub-region targeted by the immunogenic fusion peptides described herein.

FIG. 2 shows KQPA-NPDP-NANPNVDP3-NANP5-Hpferr-PADRE (126) design, expression, purification, and validation.

FIG. 3 shows KQPA-NPDP-NANPNVDP3-NANP18-Hpferr-PADRE (127) design, expression, purification, and validation.

FIG. 4 shows KQPA-NPDP-NANPNVDP3-NANP5-LS-PADRE (128) design, expression, purification, and validation.

FIG. 5 shows KQPA-NPDP-NANPNVDP3-NANP18-LS-PADRE (129) design, expression, purification, and validation.

FIG. 6 shows the immunization experimental design in WT mice.

FIG. 7 shows serum antibody responses to different PfCSP peptides.

FIG. 8 shows T cell responses to different PfCSP peptides.

FIG. 9 shows glycan masking of PfCSP antigens.

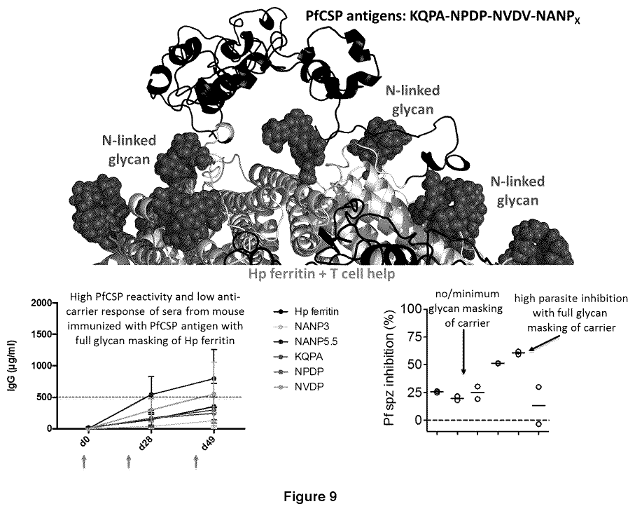

FIG. 10. Protection of mice immunized according to prime-boost-boost scheme (10 μg of the indicated immunogens in Adjuvant 1) against infections with rodent malaria parasites expressing human malaria parasite CSP (Pb-PfCSP) by direct mosquito bites (three infectious bites per mouse). Control group (Adjuvant 1) received adjuvant following the same immunization scheme. Data show two independent experiments (n=10 mice per condition).

FIG. 11. Protection of mice immunized according to the prime (d0)-boost (d28) scheme (10 or 0.5 μg of the indicated immunogens in Adjuvant 2) against infections with rodent malaria parasites expressing human malaria parasite CSP (Pb-PfCSP) by direct mosquito bites (three infectious bites per mouse). Control group (Adjuvant 2) received adjuvant only following the same immunization scheme. Data show one experiment (n=5 mice per condition).

FIG. 12. Anti-full-length CSP (FL-CSP), NANP-5 and KQPA peptide IgG concentrations at d35 in sera of mice immunized with the indicated immunogens in Adjuvant 2 or with Adjuvant 2 alone following a prime (d0)-booster (d28) immunization scheme. Symbols show individual mice. Horizontal bars indicate means. Data show one experiment (n=5 mice per group).

### DETAILED DESCRIPTION

### Definitions

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

It is also to be understood that the terminology used herein is for the purpose of describing particular aspects only and is not intended to be limiting. Many patent applications, patents, and publications are referred to herein to assist in understanding the aspects described. Each of these references are incorporated herein by reference in their entirety.

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Typically, a composition consisting essentially of a set of components will comprise less than 5% by weight, typically less than 3% by weight, more typically less than 1%, and even more typically less than 0.1% by weight of non-specified component(s).

It will be understood that any component defined herein as being included may be explicitly excluded from the claimed invention by way of proviso or negative limitation. For example, the fusion proteins described herein, in aspects, exclude the C-terminal domain of PfCSP.

In addition, all ranges given herein include the end of the ranges and also any intermediate range points, whether explicitly stated or not. With respect to the motifs described herein, which contain four amino acids, it will be understood that ranges of, for example, 0-20 repeats of these motifs encompass quarter integers as well, for example, 0.25, 0.5, and 0.75, and all other integer quarters, are contemplated herein. This indicates that the repeat is not necessarily complete and, for example, NANP may be repeated as NANPN, NANPNA, NANPNAN, NANPNANP, N, NA, NAN, NANP, A, AN, ANP, ANPN, N, NP, NPN, NPNA, P, PN, PNA, or PNAN, and so on.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.” The word “or” is intended to include “and” unless the context clearly indicates otherwise.

The terms “protein nanoparticle” and “nanocage” are used interchangeably herein and refer to a multi-subunit, protein-based polyhedron shaped structure. The subunits or nanocage monomers are each composed of proteins or polypeptides (for example a glycosylated polypeptide), and, optionally of single or multiple features of the following: nucleic acids, prosthetic groups, organic and inorganic compounds. Non-limiting examples of protein nanoparticles include ferritin nanoparticles (see, e.g., Zhang, Y. Int. J. Mol. Sci., 12:5406-5421, 2011, incorporated by reference herein), encapsulin nanoparticles (see, e.g., Sutter et al., Nature Struct, and Mol. Biol., 15:939-947, 2008, incorporated by reference herein), Sulfur Oxygenase Reductase (SOR) nanoparticles (see, e.g., Urich et al., Science, 311:996-1000, 2006, incorporated by reference herein), lumazine synthase nanoparticles (see, e.g., Zhang et al., J. Mol. Biol., 306: 1099-1114, 2001) or pyruvate dehydrogenase nanoparticles (see, e.g., Izard et al., PNAS 96: 1240-1245, 1999, incorporated by reference herein). Ferritin, apoferritin, encapsulin, SOR, lumazine synthase, and pyruvate dehydrogenase are monomeric proteins that self-assemble into a globular protein complexes that in some cases consists of 24, 60, 24, 60, and 60 protein subunits, respectively. Ferritin and apoferritin are generally referred to interchangeably herein and are understood to both be suitable for use in the fusion proteins, nanocages, and methods described herein. Carboxysome, vault proteins, GroEL, heat shock protein, E2P and MS2 coat protein also produce nanocages are contemplated for use herein. In addition, fully or partially synthetic self-assembling monomers are also contemplated for use herein.

It will be understood that each nanocage monomer may be divided into two or more subunits that will self-assemble into a functional nanocage monomer. For example, ferritin or apoferritin may be divided into an N- and C-subunit, divided substantially in half, so that each subunit may be separately bound to a different bioactive moiety for subsequent self-assembly into a nanocage monomer and then a nanocage. By “functional nanocage monomer” it is intended that the nanocage monomer is capable of self-assembly with other such monomers into a nanocage as described herein.

A “vaccine” is a pharmaceutical composition that induces a prophylactic or therapeutic immune response in a subject. In some cases, the immune response is a protective immune response. Typically, a vaccine induces an antigen-specific immune response to an antigen of a pathogen, for example a viral pathogen, or to a cellular constituent correlated with a pathological condition. A vaccine may include a polynucleotide (such as a nucleic acid encoding a disclosed antigen), a peptide or polypeptide (such as a disclosed antigen), a virus, a cell or one or more cellular constituents. In one specific, non-limiting example, a vaccine induces an immune response that reduces the severity of the symptoms associated with malaria infection and/or decreases the parasite load compared to a control. In another non-limiting example, a vaccine induces an immune response that reduces and/or prevents malaria infection compared to a control.

The term “antibody”, also referred to in the art as “immunoglobulin” (Ig), used herein refers to a protein constructed from paired heavy and light polypeptide chains, various Ig isotypes exist, including IgA, IgD, IgE, IgG, and IgM. When an antibody is correctly folded, each chain folds into a number of distinct globular domains joined by more linear polypeptide sequences. For example, the immunoglobulin light chain folds into a variable (V_{L}) and a constant (CL) domain, while the heavy chain folds into a variable (V_{H}) and three constant (C_{H}, C_{H2}, C_{H3}) domains. Interaction of the heavy and light chain variable domains (V_{H} and V_{L}) results in the formation of an antigen binding region (Fv). Each domain has a well-established structure familiar to those of skill in the art.

The light and heavy chain variable regions are responsible for binding the target antigen and can therefore show significant sequence diversity between antibodies. The constant regions show less sequence diversity, and are responsible for binding a number of natural proteins to elicit important immunological events. The variable region of an antibody contains the antigen binding determinants of the molecule, and thus determines the specificity of an antibody for its target antigen.

The majority of sequence variability occurs in six hypervariable regions, three each per variable heavy and light chain; the hypervariable regions combine to form the antigen-binding site, and contribute to binding and recognition of an antigenic determinant. The specificity and affinity of an antibody for its antigen is determined by the structure of the hypervariable regions, as well as their size, shape and chemistry of the surface they present to the antigen.

An “antibody fragment” as referred to herein may include any suitable antigen-binding antibody fragment known in the art. The antibody fragment may be a naturally-occurring antibody fragment, or may be obtained by manipulation of a naturally-occurring antibody or by using recombinant methods. For example, an antibody fragment may include, but is not limited to a Fv, single-chain Fv (scFv; a molecule consisting of V_{L} and V_{H} connected with a peptide linker), Fab, F(ab′)₂, single domain antibody (sdAb; a fragment composed of a single V_{L} or V_{H}), and multivalent presentations of any of these.

By the term “synthetic antibody” as used herein, is meant an antibody which is generated using recombinant DNA technology. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “epitope” refers to an antigenic determinant. An epitope is the particular chemical groups or peptide sequences on a molecule that are antigenic, that is, that elicit a specific immune response. An antibody specifically binds a particular antigenic epitope, e.g., on a polypeptide. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, about 11, or about 8 to about 12 amino acids in a unique spatial conformation. Methods of determining spatial conformation of epitopes include, for example, x-ray crystallography and 2-dimensional nuclear magnetic resonance. See, e.g., “Epitope Mapping Protocols” in Methods in Molecular Biology, Vol. 66, Glenn E. Morris, Ed (1996).

The term “antigen” and “immunogenic peptide” are used interchangeably herein and as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the aspects described herein include, but are not limited to, the use of partial nucleotide sequences of more than one gene and that these nucleotide sequences could be arranged in various combinations to elicit the desired immune response. Moreover, a skilled artisan will understand that an antigen need not be encoded by a “gene” at all. It is readily apparent that an antigen can be synthesized or can be derived from a biological sample. Such a biological sample can include, but is not limited to a tissue sample, a cell, or a biological fluid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, typically, a human.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species, for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The terms “therapeutically effective amount”, “effective amount” or “sufficient amount” mean a quantity sufficient, when administered to a subject, including a mammal, for example a human, to achieve a desired result, for example an amount effective to cause a protective immune response. Effective amounts of the compounds described herein may vary according to factors such as the immunogen, age, sex, and weight of the subject. Dosage or treatment regimes may be adjusted to provide the optimum therapeutic response, as is understood by a skilled person. For example, administration of a therapeutically effective amount of the fusion proteins described herein is, in aspects, sufficient to increase immunity against a pathogen, such as Plasmodium.

Moreover, a treatment regime of a subject with a therapeutically effective amount may consist of a single administration, or alternatively comprise a series of applications. The length of the treatment period depends on a variety of factors, such as the immunogen, the age of the subject, the concentration of the agent, the responsiveness of the patient to the agent, or a combination thereof. It will also be appreciated that the effective dosage of the agent used for the treatment may increase or decrease over the course of a particular treatment regime. Changes in dosage may result and become apparent by standard diagnostic assays known in the art. The fusion proteins described herein may, in aspects, be administered before, during or after treatment with conventional therapies for the disease or disorder in question, such as malaria.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “subject” as used herein refers to any member of the animal kingdom, typically a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

Administration “in combination with” one or more further therapeutic agents includes simultaneous (concurrent) and consecutive administration in any order.

The term “pharmaceutically acceptable” means that the compound or combination of compounds is compatible with the remaining ingredients of a formulation for pharmaceutical use, and that it is generally safe for administering to humans according to established governmental standards, including those promulgated by the United States Food and Drug Administration.

The term “pharmaceutically acceptable carrier” includes, but is not limited to solvents, dispersion media, coatings, antibacterial agents, antifungal agents, isotonic and/or absorption delaying agents and the like. The use of pharmaceutically acceptable carriers is well known.

The term “adjuvant” refers to a compound or mixture that is present in a vaccine and enhances the immune response to an antigen present in the vaccine. For example, an adjuvant may enhance the immune response to a polypeptide present in a vaccine as contemplated herein, or to an immunogenic fragment or variant thereof as contemplated herein. An adjuvant can serve as a tissue depot that slowly releases the antigen and also as a lymphoid system activator that non-specifically enhances the immune response. Examples of adjuvants which may be employed include MPL-TDM adjuvant (monophosphoryl Lipid A/synthetic trehalose dicorynomycolate, e.g., available from GSK Biologics). Another suitable adjuvant is the immunostimulatory adjuvant AS01/AS02 (GSK). These immunostimulatory adjuvants are formulated to give a strong T cell response and include QS-21, a saponin from Quillay Saponaria, the TL4 ligand, a monophosphoryl lipid A, together in a lipid or liposomal carrier. Other adjuvants include, but are not limited to, nonionic block co-polymer adjuvants (e.g., CRL 1005), aluminum phosphates (e.g., AIPO.sub.4), R-848 (a Th1-like adjuvant), imiquimod, PAM3CYS, poly (1:C), loxoribine, BCG (bacille Calmette-Guerin) and Corynebacterium parvum, CpG oligodeoxynucleotides (ODN), cholera toxin derived antigens (e.g., CTA 1-DD), lipopolysaccharide adjuvants, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions in water (e.g., MF59 available from Novartis Vaccines or Montanide ISA 720), keyhole limpet hemocyanins, and dinitrophenol.

“Variants” are biologically active fusion proteins, antibodies, or fragments thereof having an amino acid sequence that differs from a comparator sequence by virtue of an insertion, deletion, modification and/or substitution of one or more amino acid residues within the comparative sequence. Variants generally have less than 100% sequence identity with the comparative sequence. Ordinarily, however, a biologically active variant will have an amino acid sequence with at least about 70% amino acid sequence identity with the comparative sequence, such as at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity. The variants include peptide fragments of at least 10 amino acids that retain some level of the biological activity of the comparator sequence. Variants also include polypeptides wherein one or more amino acid residues are added at the N- or C-terminus of, or within, the comparative sequence. Variants also include polypeptides where a number of amino acid residues are deleted and optionally substituted by one or more amino acid residues. Variants also may be covalently modified, for example by substitution with a moiety other than a naturally occurring amino acid or by modifying an amino acid residue to produce a non-naturally occurring amino acid.

“Percent amino acid sequence identity” is defined herein as the percentage of amino acid residues in the candidate sequence that are identical with the residues in the sequence of interest, such as the polypeptides of the invention, after aligning the sequences and introducing gaps, if necessary, to achieve the maximum percent sequence identity, and not considering any conservative substitutions as part of the sequence identity. None of N-terminal, C-terminal, or internal extensions, deletions or insertions into the candidate sequence shall be construed as affecting sequence identity or homology. Methods and computer programs for the alignment are well known in the art, such as “BLAST”.

“Active” or “activity” for the purposes herein refers to a biological and/or an immunological activity of the fusion proteins described herein, wherein “biological” activity refers to a biological function (either inhibitory or stimulatory) caused by the fusion proteins.

The fusion proteins described herein may include modifications. Such modifications include, but are not limited to, conjugation to an effector molecule such as an anti-malaria agent or an adjuvant. Modifications further include, but are not limited to conjugation to detectable reporter moieties. Modifications that extend half-life (e.g., pegylation) are also included. Proteins and non-protein agents may be conjugated to the fusion proteins by methods that are known in the art. Conjugation methods include direct linkage, linkage via covalently attached linkers, and specific binding pair members (e.g., avidin-biotin). Such methods include, for example, that described by Greenfield et al., Cancer Research 50, 6600-6607 (1990), which is incorporated by reference herein and those described by Amon et al., Adv. Exp. Med. Biol. 303, 79-90 (1991) and by Kiseleva et al, Mol. Biol. (USSR)25, 508-514 (1991), both of which are incorporated by reference herein.

A “PanDR binding” peptide or “PADRE®” peptide (Epimmune, San Diego, Calif.) is a member of a family of molecules that binds more than one HLA class II DR molecule. The pattern that defines the PADRE® family of molecules can be referred to as an HLA Class II supermotif. A PADRE® molecule binds to HLA-DR molecules and stimulates in vitro and in vivo human helper T lymphocyte (HTL) responses and can be referred to as providing exogenous T cell help. For a further definition of the PADRE® family, see for example, U.S. Ser. No. 09/709,774; Ser. No. 09/707,738; PCT publication Nos WO 95/07707, and WO 97/26784; U.S. Pat. Nos. 5,736,142; 5,679,640; and 6,413,935, each of which is incorporated herein by reference in its entirety.

Fusion Proteins Described herein are fusion proteins. Typically, the fusion proteins comprise one or more motifs derived from PfCSP. For example, the immunogenic fusion protein in aspects comprises at least one of the following motifs: KQPA, NPDP, NANP, NVDP, and NANP, which can be present in any order and repeated in order or not to any extent. The fusion proteins described herein are immunogenic and find use in the treatment and/or prevention of malaria.

In certain aspects, the fusion proteins described herein comprise an immunogenic peptide fused to a nanocage monomer peptide, either directly or via a linker or other moiety. The immunogenic peptide comprises one or more repeat motifs derived from PfCSP, such as:
KQPAₐ; QPAKₐ; PAKQₐ; or AKQPₐ;
NPDP_{b}; PDPN_{b}; DPNP_{b}; or PNPD_{b};
NANP_{c}; ANPN_{c}; NPNA_{c}; or PNAN_{c};
NVDP_{d}; VDPN_{d}; DPNV_{d}; or PNVD_{d}; and
NANPₑ; ANPNₑ; NPNAₑ; or PNANₑ.

The letters a, b, c, d, and e designate how many times the given motif is repeated and each of a, b, c, d, and e are independently present or absent and, if present, can be repeated any desired number of times as long as the resultant fusion protein remains immunogenic. Typically, at least two motifs are present, such that a+b+c+d+e is at least 2.

Typically a, b, c, d, e, or any combination thereof are each independently at least about 1 and more typically, a, b, c, d, e, or any combination thereof are each independently from about 1 to about 40. This means that a, b, c, d, and e are each usually present and are each typically either not repeated or repeated up to about 40 times. Fractional repeats are understood to be included herein, as each motif comprising 4 amino acid, therefore a 1.25 repeat would be understood to include the original motif with the first amino acid repeated, for example, NANPN. Likewise, a 1.5 repeat would represent for example NANPNA, and a 1.75 repeat would represent for example NANPNAN.

From the above, it will be understood that a, b, c, d, and e are each independently present or absent and optionally repeated any number of times. Typically, however, each of a, b, c, d, and e are each present and are independently from about 1 to about 100, such as from about 1, about 2, about 3, about 4, about 5, about 6, about 7, about 8, about 9, about 10, about 11, about 12, about 13, about 14, about 15, about 16, about 17, about 18, about 19, or about 20 to about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 60, about 70, about 75, about 80, about 90, or about 100, such as from about 1 to about 40, or about 1 to about 20, or from 1 to about 10. Typically, a is 1, b is 1, c is 1, d is 3, and e is 5 or 18.5.

It will be understood that when any given motif is repeated in whole or in part, i.e., when a, b, c, d, or e is greater than 1, the repeated motif may be contiguous or non-contiguous with the original motif. For example, for NANP2, this may be NANPNANP or NANP-intervening sequence-NANP. There may be combinations of contiguous or non-contiguous repeated motifs as well, such as, for example, NANPNANP-intervening sequence-NANP.

It will be understood that the motifs listed above and their respect repeats if present may be in any order, however, the motifs are typically in the order KQPAₐ-NPDP_{b}-NANP_{c}-NVDP_{d}-NANPₑ.

The variant sequences or fragments described herein may have any desired sequence identity to the comparator sequences herein, as long as they retain at least some level of the desired function of the comparator sequence. For example, the fusion peptides described herein are immunogenic and variants of these peptides would retain at least some immunogenicity. Typically, variants comprise at least about 70%, 75%, 80%, 85%, 90%, 95%, 97%, or 99% sequence identity to the immunogenic peptide.

The nanocage monomers described herein can be any of the nanocage monomers as described in, for example, WO/2019/023811, which is incorporated herein by reference. Typically, the nanocage monomer is ferritin, apoferritin, encapsulin, SOR, lumazine synthase, pyruvate dehydrogenase, carboxysome, vault proteins, GroEL, heat shock protein, E2P, or MS2 coat protein, or fragments thereof, or variants thereof and may be provided as two or more self-assembling subunits.

The nanocage monomer may be derived from any species source, but typically is from Helicobacter pylori and is typically ferritin, termed HpFerr for short. Also typically, the nanocage monomer peptide is not human. In this way, anti-self immune responses can be mitigated.

Typically, the nanocage monomer peptide comprises or consists of the amino acid sequence:

MLSKDIIKLLNEQVNKEMNSSNLYMSMSSWCYTHSLDGAGLFLFDHAAE
EYEHAKKLIVFLNENNVPVQLTSISAPEHKFEGLTQIFQKAYEHEQHIS
ESINNIVDHAIKSKDHATFNFLQWYVAEQHEEEVLFKDILDKIELIGNE
NHGLYLADQYVKGIAKSRKS.

Similar to above, functional variants or fragments are also included. Typically, variants have at least 70% sequence identity to the reference sequence and variants and fragments are capable of self-assembly into a nanocage.

It will be understood that the nanocage monomer peptide may be modified in a variety of different ways in order to reduce an anti-nanocage monomer peptide immune response. For example, the nanocage monomer peptide may at least partially or fully masked, for example, partially or fully glycan masked. Thus, the nanocage monomer may comprise at least one NXT and/or NXS glycosylation motif. For example, the sequence noted above (or a variant or fragment thereof) may be modified to comprise a K77N and E79T mutation and/or an E99N and I101T mutations.

Typically, the nanocage monomer peptide is selected so that a plurality of the nanocage monomer peptides self-assemble into a nanocage. It will be understood that the immunogenic peptide may decorate the interior and/or exterior surface of the nanocage.

The fusion protein described herein may comprise additional peptide sequences. For example, a peptide providing exogenous T cell help and/or a peptide that provides autologous T cell help may be fused to the other peptides described herein in any order. In aspects, the peptide that provides exogenous T cell help comprises a PADRE peptide and/or a peptide derived from a pathogenic molecule, such as a tetanus toxoid peptide. If a PADRE peptide is used, it typically comprises the amino acid sequence AKFVAAWTLKAAA, or a functional variant thereof having at least 70% sequence identity thereto or a fragment of either thereof. In alternate or additional aspects, a peptide providing autologous T cell help may be included herein. Typically, the peptide that provides autologous T cell help comprises a PfCSP T cell peptide epitope.

As described above with respect to the immunogenic peptide, the peptide that provides exogenous T cell help and/or the peptide that provides autologous T cell help may independently decorate the interior and/or exterior surface of the assembled nanocage, and this may be the same or different from the way in which the immunogenic peptide decorates the nanocage.

In certain aspects, the fusion proteins described herein comprise one or more flexible or inflexible linkers between one or more of the motifs, the nanocage monomer, and any further peptides, such as the peptide that provides exogenous T cell help and/or the peptide that provide autologous T cell help. Typically, the linker is sufficiently flexible to allow the immunogenic peptide to adopt a favourable conformation, once the protein is expressed.

The linker is generally long enough to impart some flexibility to the antigen, although it will be understood that linker length will vary depending upon the antigen and antibody sequences and the three-dimensional conformation of the fusion protein. Thus, the linker is typically from about 1 to about 30 amino acid residues, such as from about 1, 2, 3, 4, 5, 6, 7, 8, 9, 10, 11, 12, 13, 14, 15, 16, 17, 18, 19, 20, 21, 22, 23, 24, 25, 26, 27, 28, or 29 to about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues, such as from about 8 to about 12 amino acid residues, such as 8, 10, or 12 amino acid residues.

The linker may be of any amino acid sequence that does not interfere with the binding of the immunogenicity of the immunogenic peptide. In one typical example, the flexible linker comprises GGS or a GGS repeat, for example, GGSGGSGGSG, GGGGSGGSGGSGGS, or GGGGGSGGSGGSGGS.

Specific examples of immunogenic fusion proteins described herein include, for example, fusion proteins comprising or consisting of the sequence:
KQPA-NPDP-NANPNVDP3-NANP5-Hpferr-PADRE;
KQPA-NPDP-NANPNVDP3-NANP18.5-Hpferr-PADRE;
KQPA-NPDP-NANPNVDP3-NANP5-LS-PADRE; and/or
KQPA-NPDP-NANPNVDP3-NANP18.5-LS-PADRE.

More specifically, certain examples include fusion proteins comprising or consisting of the amino acid sequence:

MGILPSPGMPALLSLVSLLSVLLMGCVAETGWSHPQFEKLKENLYFQGKQPADGNPDPNAN
PNVDPNANPNVDPNANPNVDPNANPNANPNANPNANPNANPSRGGGGGSGGSGGSGGSDIIKLLN
EQVNKEMQSSNLYMSMSSWCYTHSLDGAGLFLFDHAAEEYEHAKKLIIFLNENNVPVQLTSISAPEHK
FEGLTQIFQKAYEHEQHISESINNIVDHAIKSKDHATFNFLQWYVAEQHEEEVLFKDILDKIELIGNENH
GLYLADQYVKGIAKSRKSGGSASAKFVAAWTLKAAA;
MGILPSPGMPALLSLVSLLSVLLMGCVAETGWSHPQFEKLKENLYFQGKQPADGNPDPNAN
PNVDPNANPNVDPNANPNVDPNANPNANPNANPNANPNANPNANPNANPNANPNANPNANPNANP
NANPNANPNANPNANPNANPNANPNANPSRGGGGGSGGSGGSGGSDIIKLLNEQVNKEMQSSNLY
MSMSSWCYTHSLDGAGLFLFDHAAEEYEHAKKLIIFLNENNVPVQLTSISAPEHKFEGLTQIFQKAYEH
EQHISESINNIVDHAIKSKDHATFNFLQWYVAEQHEEEVLFKDILDKIELIGNENHGLYLADQYVKGIAK
SRKSGGSASAKFVAAWTLKAAA;
MGILPSPGMPALLSLVSLLSVLLMGCVAETGWSHPQFEKLKENLYFQGKQPADGNPDPNAN
PNVDPNANPNVDPNANPNVDPNANPNANPNANPNANPNANPSRGGGGGSGGSGGSGGSMQIYEG
KLTAEGLRFGIVASRFNHALVDRLVEGAIDAIVRHGGREEDITLVRVPGSWEIPVAAGELARKEDIDAVI
AIGVLIRGATPHFDYIASEVSKGLADLSLELRKPITFGVITADTLEQAIERAGTKHGNKGWEAALSAIEM
ANLFKSLRGGSASAKFVAAWTLKAAA;
MGILPSPGMPALLSLVSLLSVLLMGCVAETGWSHPQFEKLKENLYFQGKQPADGNPDPNAN
PNVDPNANPNVDPNANPNVDPNANPNANPNANPNANPNANPNANPNANPNANPNANPNANPNANP
NANPNANPNANPNANPNANPNANPNANPSRGGGGGSGGSGGSGGSMQIYEGKLTAEGLRFGIVAS
RFNHALVDRLVEGAIDAIVRHGGREEDITLVRVPGSWEIPVAAGELARKEDIDAVIAIGVLIRGATPHFD
YIASEVSKGLADLSLELRKPITFGVITADTLEQAIERAGTKHGNKGWEAALSAIEMANLFKSLRGGSAS
AKFVAAWTLKAAA;
and/or
MGILPSPGMPALLSLVSLLSVLLMGCVAETGWSHPQFEKLKENLYFQGKQPADGNPDPNAN
PNVDPNANPNVDPNANPNVDPNANPNANPNANPNANPNANPNANPNANPNANPNANPNANPNANP
NANPNANPNANPNANPNANPNANPNANPSRGGGGGSGGSGGSGGSDIIKLLNEQVNKEMQSSNLY
MSMSSWCYTHSLDGAGLFLFDHAAEEYEHAKKLIIFLNENNVPVQLTSISAPEHNFTGLTQIFQKAYEH
EQHISNSTNNIVDHAIKSKDHATFNFLQWYVAEQHEEEVLFKDILDKIELIGNENHGLYLADQYVKGIAK
SRKSGGSASAKFVAAWTLKAAA;

or functional variants or fragments thereof.

Also described herein are nucleic acid molecules encoding the fusion proteins described herein, vectors, host cells, and vaccines comprising the fusion proteins described herein. As will be understood, vaccines may include, for example, adjuvants, as further described above.

The fusion proteins described herein are immunogenic and are capable of eliciting an immune response in a subject. Thus, in aspects, antibodies that bind to the fusion proteins described herein are also contemplated. Methods of immunizing subjects, including humans and animals, in order to produce and characterize such antibodies are known. Such antibodies can then be used in assays, therapeutic or preventative compositions, etc.

The fusion proteins described herein are, in aspects, useful as a highly efficacious pre-erythrocytic subunit malaria vaccine. These are also or alternatively, in aspects, a C-terminal truncated PfCSP antigen. In addition or alternately, the fusion proteins described herein are useful in providing a boostable malaria vaccine that is PfCSP-based.

Further described herein is a glycan-masked nanocage monomer peptide, which may be as described above. For example, the nanocage monomer is typically ferritin, apoferritin, encapsulin, SOR, lumazine synthase, pyruvate dehydrogenase, carboxysome, vault proteins, GroEL, heat shock protein, E2P, or MS2 coat protein, or fragments thereof, or variants thereof. The nanocage monomer may be provided as two or more self-assembling subunits. Typically, the nanocage monomer peptide is from Helicobacter pylori and/or is not human.

It will be understood that the nanocage monomer peptide may further comprise a bioactive moiety, such as an antibody or fragment thereof, an antigen, a detectable moiety, a pharmaceutical agent, a diagnostic agent, or combinations thereof. Typically, the bioactive moiety comprises an antigen.

A plurality of the nanocage monomer peptides typically self-assemble into a nanocage and the bioactive moiety decorates the interior and/or exterior surface of the nanocage.

As described above, the nanocage monomer peptide is at least partially or fully masked, typically glycan masked. Generally, the nanocage monomer comprises at least one NXT and/or NXS glycosylation motif and/or comprises the amino acid sequence:

MLSKDIIKLLNEQVNKEMNSSNLYMSMSSWCYTHSLDGAGLFLFDHA
AEEYEHAKKLIVFLNENNVPVQLTSISAPEHKFEGLTQIFQKAYEHE
QHISESINNIVDHAIKSKDHATFNFLQWYVAEQHEEEVLFKDILDKI
ELIGNENHGLYLADQYVKGIAKSRKS

or a functional variant having at least 70% sequence identity thereto, or a functional fragment of either thereof, and wherein the sequence comprises a K77N and E79T mutation and/or an E99N and I101T mutations.

It will be understood that the fusion proteins may be modified as described above as a general concept and/or in the interest of immuno-modulation or immuno-focusing. Further, T-cell epitope linear peptides may be included that help immuno-modulate/increase humoral/antibody responses.

As described herein, a substantially identical sequence may comprise one or more conservative amino acid mutations. It is known in the art that one or more conservative amino acid mutations to a reference sequence may yield a mutant peptide with no substantial change in physiological, chemical, or functional properties compared to the reference sequence; in such a case, the reference and mutant sequences would be considered “substantially identical” polypeptides. Conservative amino acid mutation may include addition, deletion, or substitution of an amino acid; a conservative amino acid substitution is defined herein as the substitution of an amino acid residue for another amino acid residue with similar chemical properties (e.g. size, charge, or polarity).

In a non-limiting example, a conservative mutation may be an amino acid substitution. Such a conservative amino acid substitution may substitute a basic, neutral, hydrophobic, or acidic amino acid for another of the same group. By the term “basic amino acid” it is meant hydrophilic amino acids having a side chain pK value of greater than 7, which are typically positively charged at physiological pH. Basic amino acids include histidine (His or H), arginine (Arg or R), and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of Eisenberg (1984). Hydrophobic amino acids include proline (Pro or P), isoleucine (Ile or I), phenylalanine (Phe or F), valine (Val or V), leucine (Leu or L), tryptophan (Trp or W), methionine (Met or M), alanine (Ala or A), and glycine (Gly or G). “Acidic amino acid” refers to hydrophilic amino acids having a side chain pK value of less than 7, which are typically negatively charged at physiological pH. Acidic amino acids include glutamate (Glu or E), and aspartate (Asp or D).

Sequence identity is used to evaluate the similarity of two sequences; it is determined by calculating the percent of residues that are the same when the two sequences are aligned for maximum correspondence between residue positions. Any known method may be used to calculate sequence identity; for example, computer software is available to calculate sequence identity. Without wishing to be limiting, sequence identity can be calculated by software such as NCBI BLAST2 service maintained by the Swiss Institute of Bioinformatics (and as found at ca.expasy.org/tools/blast/), BLAST-P, Blast-N, or FASTA-N, or any other appropriate software that is known in the art.

The substantially identical sequences of the present invention may be at least 85% identical; in another example, the substantially identical sequences may be at least 70, 75, 80, 85, 90, 95, 96, 97, 98, 99, or 100% (or any percentage there between) identical at the amino acid level to sequences described herein. In specific aspects, the substantially identical sequences retain the activity and specificity of the reference sequence. In a non-limiting embodiment, the difference in sequence identity may be due to conservative amino acid mutation(s).

The polypeptides or fusion proteins of the present invention may also comprise additional sequences to aid in their expression, detection or purification. Any such sequences or tags known to those of skill in the art may be used. For example, and without wishing to be limiting, the fusion proteins may comprise a targeting or signal sequence (for example, but not limited to ompA), a detection tag, exemplary tag cassettes include Strep tag, or any variant thereof; see, e.g., U.S. Pat. No. 7,981,632, His tag, Flag tag having the sequence motif DYKDDDDK, Xpress tag, Avi tag, Calmodulin tag, Polyglutamate tag, HA tag, Myc tag, Nus tag, S tag, SBP tag, Softag 1, Softag 3, V5 tag, CREB-binding protein (CBP), glutathione S-transferase (GST), maltose binding protein (MBP), green fluorescent protein (GFP), Thioredoxin tag, or any combination thereof; a purification tag (for example, but not limited to a Hiss or His₆), or a combination thereof.

In another example, the additional sequence may be a biotin recognition site such as that described by Cronan et al in WO 95/04069 or Voges et al in WO/2004/076670. As is also known to those of skill in the art, linker sequences may be used in conjunction with the additional sequences or tags.

More specifically, a tag cassette may comprise an extracellular component that can specifically bind to an antibody with high affinity or avidity. Within a single chain fusion protein structure, a tag cassette may be located (a) immediately amino-terminal to a connector region, (b) interposed between and connecting linker modules, (c) immediately carboxy-terminal to a binding domain, (d) interposed between and connecting a binding domain (e.g., scFv) to an effector domain, (e) interposed between and connecting subunits of a binding domain, or (f) at the amino-terminus of a single chain fusion protein. In certain embodiments, one or more junction amino acids may be disposed between and connecting a tag cassette with a hydrophobic portion, or disposed between and connecting a tag cassette with a connector region, or disposed between and connecting a tag cassette with a linker module, or disposed between and connecting a tag cassette with a binding domain.

The fusion proteins may also be in a multivalent display. Multimerization may be achieved by any suitable method of known in the art. For example, and without wishing to be limiting in any manner, multimerization may be achieved using self-assembly molecules as described in Zhang et al (2004a; 2004b) and WO2003/046560.

Also encompassed herein are isolated or purified fusion proteins, polypeptides, or fragments thereof immobilized onto a surface using various methodologies; for example, and without wishing to be limiting, the polypeptides may be linked or coupled to the surface via His-tag coupling, biotin binding, covalent binding, adsorption, and the like. The solid surface may be any suitable surface, for example, but not limited to the well surface of a microtiter plate, channels of surface plasmon resonance (SPR) sensorchips, membranes, beads (such as magnetic-based or sepharose-based beads or other chromatography resin), glass, a film, or any other useful surface.

In other aspects, the fusion proteins may be linked to a cargo molecule or the assembled nanocages may hold a cargo molecule; the fusion proteins may deliver the cargo molecule to a desired site and may be linked to the cargo molecule using any method known in the art (recombinant technology, chemical conjugation, chelation, etc.). The cargo molecule may be any type of molecule, such as a therapeutic or diagnostic agent. For example, and without wishing to be limiting in any manner, the therapeutic agent may be a radioisotope, which may be used for radioimmunotherapy; a toxin, such as an immunotoxin; a cytokine, such as an immunocytokine; a cytotoxin; an apoptosis inducer; an enzyme; or any other suitable therapeutic molecule known in the art. In the alternative, a diagnostic agent may include, but is by no means limited to a radioisotope, a paramagnetic label such as gadolinium or iron oxide, a fluorophore, a Near Infra-Red (NIR) fluorochrome or dye (such as Cy3, Cy5.5, Alexa680, Dylight680, or Dylight800), an affinity label (for example biotin, avidin, etc), fused to a detectable protein-based molecule, or any other suitable agent that may be detected by imaging methods. In a specific, non-limiting example, the fusion protein may be linked to a fluorescent agent such as FITC or may genetically be fused to the Enhanced Green Fluorescent Protein (EGFP).

Antibodies against the fusion proteins described herein specifically bind to the fusion proteins. Antibody specificity, which refers to selective recognition of an antibody for a particular epitope of an antigen, for the fusion proteins described herein can be determined based on affinity and/or avidity. Affinity, represented by the equilibrium constant for the dissociation of an antigen with an antibody (K_{D}), measures the binding strength between an antigenic determinant (epitope) and an antibody binding site. Avidity is the measure of the strength of binding between an antibody with its antigen. Antibodies typically bind with a K_{D} of 10⁻⁵ to 10⁻¹¹ M. Any K_{D} greater than 10⁻⁴ M is generally considered to indicate non-specific binding. The lesser the value of the K_{D}, the stronger the binding strength between an antigenic determinant and the antibody binding site. In aspects, the antibodies described herein have a K_{D} of less than 10⁻⁴ M, 10⁻⁵ M, 10⁻⁶ M, 10⁻⁷ M, 10⁻⁸ M, or 10⁻⁹ M.

Also described herein are nucleic acid molecules encoding the fusion proteins and polypeptides described herein, as well as vectors comprising the nucleic acid molecules and host cells comprising the vectors.

Polynucleotides encoding the fusion proteins described herein include polynucleotides with nucleic acid sequences that are substantially the same as the nucleic acid sequences of the polynucleotides of the present invention. “Substantially the same” nucleic acid sequence is defined herein as a sequence with at least 70%, at least 75%, at least 80%, at least 85%, at least 90%, at least 91%, at least 92%, at least 93%, at least 94%, at least 95% identity to another nucleic acid sequence when the two sequences are optimally aligned (with appropriate nucleotide insertions or deletions) and compared to determine exact matches of nucleotides between the two sequences.

Suitable sources of DNAs that encode fragments of antibodies include any cell, such as hybridomas, that express the full-length antibody. The fragments may be used by themselves as antibody equivalents, or may be recombined into equivalents, as described above. The DNA deletions and recombinations described in this section may be carried out by known methods, such as those described in the published patent applications listed above in the section entitled “Functional Equivalents of Antibodies” and/or other standard recombinant DNA techniques, such as those described below. Another source of DNAs are single chain antibodies produced from a phage display library, as is known in the art.

Additionally, the expression vectors are provided containing the polynucleotide sequences previously described operably linked to an expression sequence, a promoter and an enhancer sequence. A variety of expression vectors for the efficient synthesis of antibody polypeptide in prokaryotic, such as bacteria and eukaryotic systems, including but not limited to yeast and mammalian cell culture systems have been developed. The vectors of the present invention can comprise segments of chromosomal, non-chromosomal and synthetic DNA sequences.

Any suitable expression vector can be used. For example, prokaryotic cloning vectors include plasmids from E. coli, such as colEI, pCRI, pBR322, pMB9, pUC, pKSM, and RP4. Prokaryotic vectors also include derivatives of phage DNA such as M13 and other filamentous single-stranded DNA phages. An example of a vector useful in yeast is the 2p plasmid. Suitable vectors for expression in mammalian cells include well-known derivatives of SV-40, adenovirus, retrovirus-derived DNA sequences and shuttle vectors derived from combination of functional mammalian vectors, such as those described above, and functional plasmids and phage DNA.

Additional eukaryotic expression vectors are known in the art (e.g., P J. Southern & P. Berg, J. Mol. Appl. Genet, 1:327-341 (1982); Subramani et al, Mol. Cell. Biol, 1: 854-864 (1981); Kaufinann & Sharp, “Amplification And Expression of Sequences Cotransfected with a Modular Dihydrofolate Reductase Complementary DNA Gene,” J. Mol. Biol, 159:601-621 (1982); Kaufhiann & Sharp, Mol. Cell. Biol, 159:601-664 (1982); Scahill et al., “Expression And Characterization Of The Product Of A Human Immune Interferon DNA Gene In Chinese Hamster Ovary Cells,” Proc. Nat'l Acad. Sci USA, 80:4654-4659 (1983); Urlaub & Chasin, Proc. Nat'l Acad. Sci USA, 77:4216-4220, (1980), all of which are incorporated by reference herein).

The expression vectors typically contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the trp system, the tac system, the trc system, major operator and promoter regions of phage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha-mating factors, and promoters derived from polyoma, adenovirus, retrovirus, and simian virus, e.g., the early and late promoters or SV40, and other sequences known to control the expression of genes of prokaryotic or eukaryotic cells and their viruses or combinations thereof.

Also described herein are recombinant host cells containing the expression vectors previously described. The fusion proteins described herein can be expressed in cell lines other than in hybridomas. Nucleic acids, which comprise a sequence encoding a polypeptide according to the invention, can be used for transformation of a suitable mammalian host cell.

Cell lines of particular preference are selected based on high level of expression, constitutive expression of protein of interest and minimal contamination from host proteins. Mammalian cell lines available as hosts for expression are well known in the art and include many immortalized cell lines, such as but not limited to, Chinese Hamster Ovary (CHO) cells, Baby Hamster Kidney (BHK) cells and many others. Suitable additional eukaryotic cells include yeast and other fungi. Useful prokaryotic hosts include, for example, E. coli, such as E. coli SG-936, E. coli HB 101, E. coli W3110, E. coli X1776, E. coli X2282, E. coli DHI, and E. coli MRC1, Pseudomonas, Bacillus, such as Bacillus subtilis, and Streptomyces.

These present recombinant host cells can be used to produce fusion proteins by culturing the cells under conditions permitting expression of the polypeptide and purifying the polypeptide from the host cell or medium surrounding the host cell. Targeting of the expressed polypeptide for secretion in the recombinant host cells can be facilitated by inserting a signal or secretory leader peptide-encoding sequence (See, Shokri et al, (2003) Appl Microbiol Biotechnol. 60(6): 654-664, Nielsen et al, Prot. Eng., 10:1-6 (1997); von Heinje et al., Nucl. Acids Res., 14:4683-4690 (1986), all of which are incorporated by reference herein) at the 5′ end of the antibody-encoding gene of interest. These secretory leader peptide elements can be derived from either prokaryotic or eukaryotic sequences. Accordingly suitably, secretory leader peptides are used, being amino acids joined to the N-terminal end of a polypeptide to direct movement of the polypeptide out of the host cell cytosol and secretion into the medium.

The fusion proteins described herein can be fused to additional amino acid residues. Such amino acid residues can be a peptide tag to facilitate isolation, for example. Other amino acid residues for homing of the fusion proteins to specific organs or tissues are also contemplated.

In another aspect, described herein are methods of vaccinating subjects by administering a therapeutically effective amount of the fusion proteins described herein to a mammal in need thereof, typically a young, juvenile, or neonatal mammal. Therapeutically effective means an amount effective to produce the desired therapeutic effect, such as providing a protective immune response against the antigen in question.

Any suitable method or route can be used to administer the fusion proteins and vaccines described herein. Routes of administration include, for example, oral, intravenous, intraperitoneal, subcutaneous, or intramuscular administration.

It is understood that the fusion proteins described herein, where used in a mammal for the purpose of prophylaxis or treatment, will be administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection may, as is well known in the art, be formulated so as to provide quick, sustained or delayed release of the active ingredient after administration to the mammal.

Although human antibodies are particularly useful for administration to humans, they may be generated using the fusion proteins described herein for administration to other mammals as well. The term “mammal” as used herein is intended to include, but is not limited to, humans, laboratory animals, domestic pets and farm animals.

Also included herein are kits for vaccination, comprising a therapeutically or prophylactically effective amount of a fusion protein described herein. The kits can further contain any suitable adjuvant for example. Kits may include instructions.

The above disclosure generally describes the present invention. A more complete understanding can be obtained by reference to the following specific examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

The following examples do not include detailed descriptions of conventional methods, such as those employed in the construction of vectors and plasmids, the insertion of genes encoding polypeptides into such vectors and plasmids, or the introduction of plasmids into host cells. Such methods are well known to those of ordinary skill in the art and are described in numerous publications including Sambrook, J., Fritsch, E. F. and Maniatis, T. (1989), Molecular Cloning: A Laboratory Manual, 2nd edition, Cold Spring Harbor Laboratory Press, which is incorporated by reference herein.

Without further description, it is believed that one of ordinary skill in the art can, using the preceding description and the following illustrative examples, make and utilize the compounds of the present invention and practice the claimed methods. The following working examples therefore, specifically point out the typical aspects of the present invention, and are not to be construed as limiting in any way the remainder of the disclosure.

### EXAMPLES

### Example 1

Plasmodium falciparum (Pf) is the parasite that accounts for most malaria fatalities globally, but a highly efficacious pre-erythrocytic subunit vaccine remains elusive. It has now been shown that protective antibodies against malaria exist that recognize the NANP repeat of the Pf circumsporozoite protein (CSP) on the surface of the parasite in addition to KQPA, NPDP, NVDP and NANA motifs, making these antibodies cross-reactive. Our structural delineation of antibodies suggests that these PfCSP repeating motifs can be recognized by B cells precursor of protective antibodies (vaccine targets) and that binding cross-reactivity to these different motifs is associated with higher overall binding affinity to PfCSP. The most potent human antibodies at inhibiting parasite infection either possess extensive cross-reactivity to different PfCSP motifs and/or high-affinity to the NANP repeat. Here, we have invented molecules that display as the B cell epitopes (Table 1) PfCSP central repeat motifs that develop cross-reactivity and high-binding affinity (including KQPA, NPDP, NVDP and NANP), and excluding the immunodominant C-terminal domain where antibody binding is not associated with parasite inhibition. These truncated PfCSP antigens are displayed on nanoparticle backbones (H. pylori apoferritin and lumazine synthase) and contain the universal T-cell epitope (Table 1) “PADRE” to effectively engage Tfh cells during B-cell maturation. We have demonstrated that these molecules express readily as secreted from mammalian cells, are well-folded and are optimally recognized in vitro by protective antibodies against malaria. In WT mice, our immunogens are the most immunogenic we have characterized against the KQPA, NPDP, NVDP and NANP PfCSP motifs, and the antibody response can be boosted in a prime-boost-boost regimen without any plateau in titers.

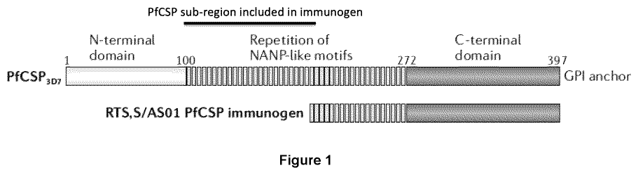

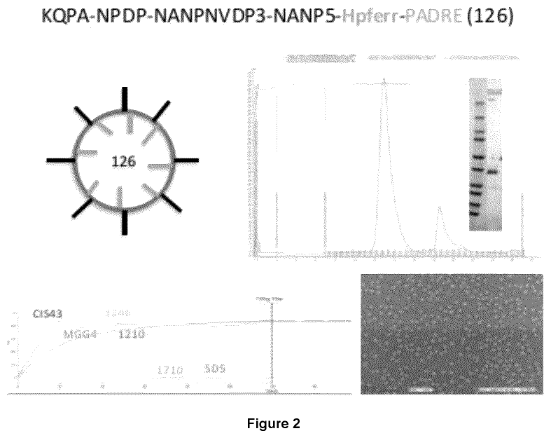

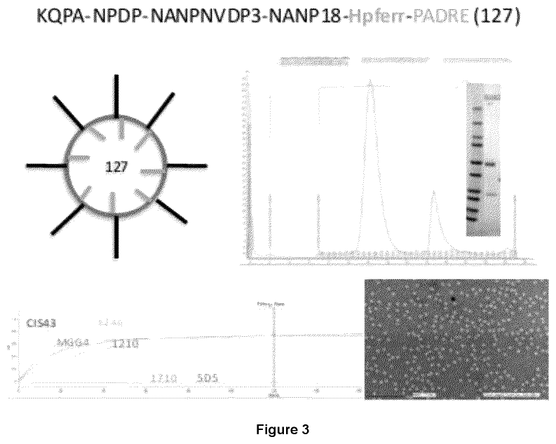

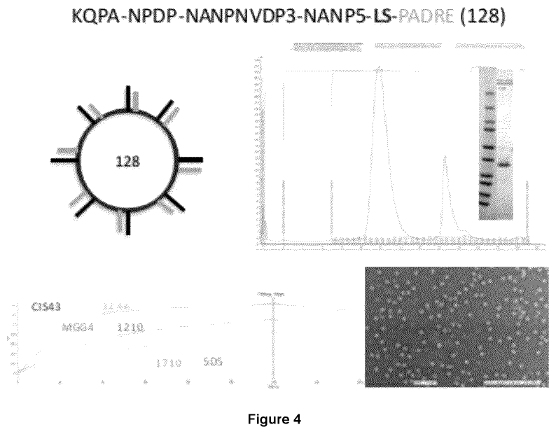

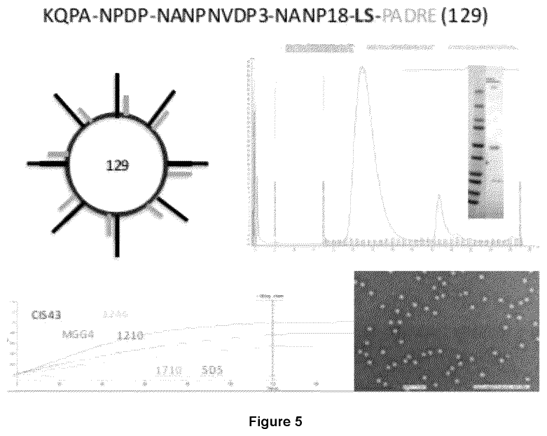

A number of different immunogenic peptides targeting the NANP-like repeat motifs of PfCSP were made and tested, as shown in FIG. 1 and Table 1. The immunogen design, expression, purification, and validation is shown in FIGS. 2-5.

TABLE 1

| | | | Protein/ | | |
|---|---|---|---|---|---|
| | Immunogen | Doses | dose (ug) | B cell epitope | T cell epitope |
| 126 | KQPA-NPDP-NANPNVDP3-NANP5-Hpferr-PADRE | 3 | 10 | | AKFVAAWTLKAAA |
| 127 | KQPA-NPDP-NANPNVDP3-NANP18-Hpferr-PADRE | 3 | 10 | | AKFVAAWTLKAAA |
| 128 | KQPA-NPDP-NANPNVDP3-NANP5-LS-PADRE | 3 | 10 | | AKFVAAWTLKAAA |
| 129 | KQPA-NPDP-NANPNVDP3-NANP18-LS-PADRE | 3 | 10 | | AKFVAAWTLKAAA |
| Ctrl | NPDPNVDPNANPNANA-Huferr-T*S | 3 | 10 | [symbol] [symbol] | [symbol] |
| | | | | [symbol] | |
| | | | | [symbol] | |
| | | | | [symbol] | |
| | | | | [symbol] | |
| Ctrl | NPNA185-Huferr-T*S | 3 | 10 | [symbol] | [symbol] |

[symbol]

indicates data missing or illegible when filed

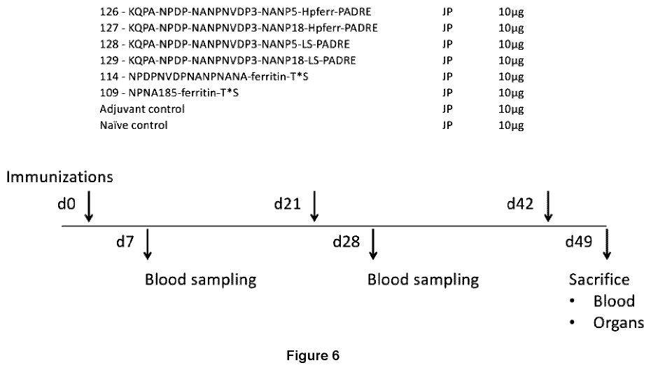

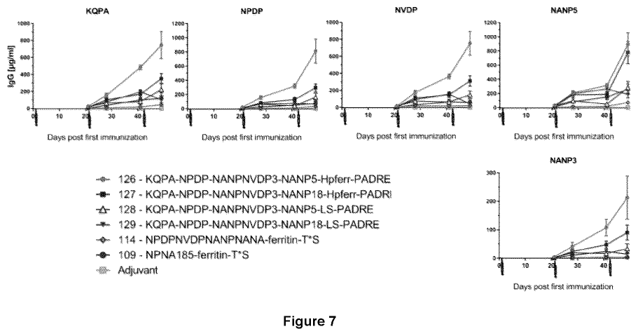

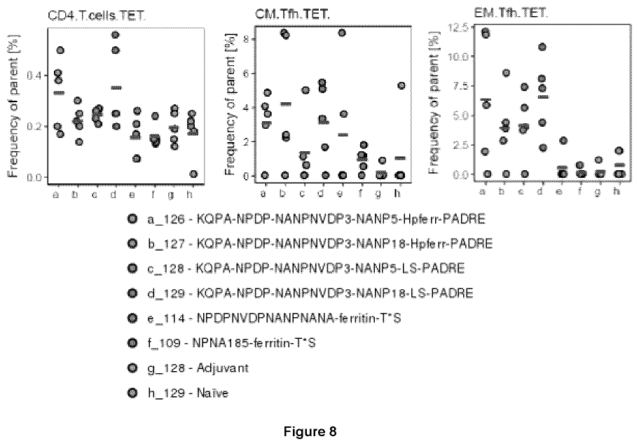

Wild-type mice were immunized using these peptides under the experimental design shown in FIG. 6. Serum antibody responses are shown in FIG. 7. It can be seen that immune responses to all peptides were observed and in some cases IgG titers reached >=1000 ug/ml. The presence of exogenous T cell help in the form of a PADRE sequence enhanced the response. These detectable PADRE T-cell responses are further shown in FIG. 8, wherein groups a and d exhibited the strongest responses.

### Example 2

### Methods

### Mice

Female C57BL/6 mice (6-7 weeks old) were purchased from a commercial vendor. Mice were immunized following a d0, d28, d70 prime-boost-boost or d0, d28 prime-boost scheme as indicated. Immunizations were two sub-cutaneous injections (100 μl total volume each, ratio of Adjuvant 1 to immunogen in buffer 1:1) left and right of the tail base for immunizations with Adjuvant 1 and two intra-muscular immunizations (25 μl total volume each, ratio Adjuvant 2 to immunogen in buffer 1:1) in the left and right thigh muscles for immunizations with Adjuvant 2. Adjuvant 1 is a Freunds-like adjuvant and Adjuvant 2 is an AS01-like adjuvant.

### Mosquitoes

Anopheles gambiae 7b line, immunocompromised transgenic mosquitoes derived from the G3 laboratory strain (Pompon et al. 2015), were kept at 28-30° C. and 70-80% humidity and used for the production of Pb-PfCSP sporozoites for in vivo infections.

### Parasites

Pb-PfCSP (eef1α:/uc+HSP70::mCherry), a replacement Plasmodium berghei (Pb) line expressing P. falciparum CSP (NF54) under the control of the Pb CSP regulatory sequences, was obtained from Chris J. Janse and Blandine M. D. Franke-Fayard and passaged every 3-4 days in CD1 female mice.

Mosquito Infections with P. berghei Parasites

A. gambiae 7b mosquitoes were fed on 8-12 weeks old female CD-1 mice infected with Pb-PfCSP parasites (0.1-0.8% gametocytemia) and kept at 20° C. and 80% humidity. Infected mosquitoes were offered an additional uninfected blood meal 7 days post infection. On day 17 after mosquito infections, infected mosquitoes were sorted by occurrence of Pb-PfCSP mCherry signal in their salivary glands using a fluorescence stereoscope (M165 C, Leica) and placed into cups.

Mouse Infections with P. berghei Parasites by Mosquito Bites

On day 18 after mosquito infections, immunized mice were individually placed on cups containing infected A. gambiae mosquitoes and the number of blood-fed mosquitoes was monitored to make sure that each immunized mouse received bites from three infected mosquitoes. From day 3 to day 7 and on day 10 after infection, blood samples were collected daily from each mouse, examined for parasitaemia by FACS, and verified by thin blood smears if necessary. All infected mice were sacrificed seven days after infection by mosquito bites.

### Results

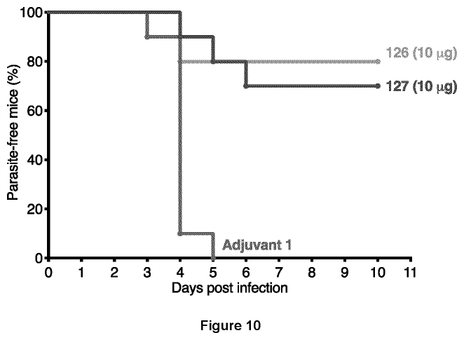

FIG. 10 shows the protection of mice immunized according to the prime-boost-boost scheme (10 μg of the indicated immunogens in Adjuvant 1) against infections with rodent malaria parasites expressing human malaria parasite CSP (Pb-PfCSP) by direct mosquito bites (three infectious bites per mouse). Control group (Adjuvant 1) received adjuvant following the same immunization scheme. Data show two independent experiments (n=10 mice per condition).

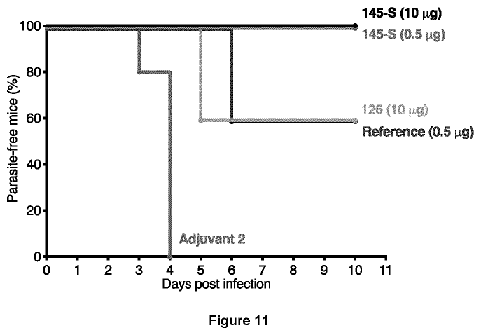

FIG. 11 shows the protection of mice immunized according to the prime (d0)-boost (d28) scheme (10 or 0.5 μg of the indicated immunogens in Adjuvant 2) against infections with rodent malaria parasites expressing human malaria parasite CSP (Pb-PfCSP) by direct mosquito bites (three infectious bites per mouse). Control group (Adjuvant 2) received adjuvant only following the same immunization scheme. Data show one experiment (n=5 mice per condition).

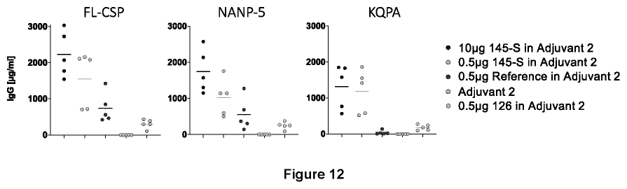

FIG. 12 shows anti-full-length CSP (FL-CSP), NANP-5 and KQPA peptide IgG concentrations at d35 in sera of mice immunized with the indicated immunogens in Adjuvant 2 or with Adjuvant 2 alone following a prime (d0)-booster (d28) immunization scheme. Symbols show individual mice. Horizontal bars indicate means. Data show one experiment (n=5 mice per group).

What is claimed is:

1. An immunogenic fusion protein comprising:
an immunogenic peptide or an immunogenic variant thereof, the immunogenic peptide comprising the following motifs:
KQPAₐ; QPAKₐ; PAKQₐ; or AKQPₐ;
NPDP_{b}; PDPN_{b}; DPNP_{b}; or PNPD_{b};
NANP_{c}; ANPN_{c}; NPNA; or PNAN_{c};
NVDP_{d}; VDPN_{d}; DPNV_{d}; or PNVD_{d}; and
NANPₑ; ANPNₑ; NPNAₑ; or PNANₑ;
wherein a, b, c, d, and e are each independently 0 or greater and wherein a+b+c+d+e is at least 2; and
a nanocage monomer peptide.

2. The fusion protein of claim 1, wherein a, b, c, d, e, or any combination thereof are each independently at least about 1.

3. The fusion protein of claim 2, wherein a, b, c, d, e, or any combination thereof are each independently from about 1 to about 40.

4. The fusion protein of claim 3, wherein a, b, c, d, and e are each independently from about 1 to about 100, such as from about 1, about 2, about 3, about 4, about 5, about 6, about 7, about 8, about 9, about 10, about 11, about 12, about 13, about 14, about 15, about 16, about 17, about 18, about 19, or about 20 to about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 60, about 70, about 75, about 80, about 90, or about 100, such as from about 1 to about 40, or about 1 to about 20, or from 1 to about 10.

5. The fusion protein of any one of claims 1 to 4, wherein a is 1.

6. The fusion protein of any one of claims 1 to 5, wherein b is 1.

7. The fusion protein of any one of claims 1 to 6, wherein c is 1.

8. The fusion protein of any one of claims 1 to 7, wherein d is 3.

9. The fusion protein of any one of claims 1 to 8, wherein e is 5.

10. The fusion protein of any one of claims 1 to 9, wherein e is 18.5.

11. The fusion protein of any one of claims 1 to 10, wherein when a, b, c, d, and/or e are greater than 1 such that the respective motif is at least partially repeated, the repeated motifs are each independently contiguous.

12. The fusion protein of any one of claims 1 to 11, wherein when a, b, c, d, and/or e are greater than 1 such that the respective motif is at least partially repeated, the repeated motifs are each independently non-contiguous.

13. The fusion protein of any one of claims 1 to 12, wherein the motifs are in the order KQPAₐ-NPDP_{b}-NANP_{c}-NVDP_{d}-NANPₑ.

14. The fusion protein of any one of claims 1 to 13, wherein the variant comprises at least about 70%, 75%, 80%, 85%, 90%, 95%, 97%, or 99% sequence identity to the immunogenic peptide.

15. The fusion protein of any one of claims 1 to 14, wherein the nanocage monomer is ferritin, apoferritin, encapsulin, SOR, lumazine synthase, pyruvate dehydrogenase, carboxysome, vault proteins, GroEL, heat shock protein, E2P, or MS2 coat protein, or fragments thereof, or variants thereof.

16. The fusion protein of claim 15, wherein the nanocage monomer is provided as two or more self-assembling subunits.

17. The fusion protein of any one of claims 1 to 16, wherein the nanocage monomer peptide is from Helicobacter pylori.

18. The fusion protein of any one of claims 1 to 16, wherein the nanocage monomer peptide is not human.

19. The fusion protein of any one of claims 1 to 18, comprising the amino acid sequence:
MLSKDIIKLLNEQVNKEMNSSNLYMSMSSWCYTHSLDGAGLFLFDHA
AEEYEHAKKLIVFLNENNVPVQLTSISAPEHKFEGLTQIFQKAYEHE
QHISESINNIVDHAIKSKDHATFNFLQWYVAEQHEEEVLFKDILDKI
ELIGNENHGLYLADQYVKGIAKSRKS
or a functional variant having at least 70% sequence identity thereto, or a functional fragment of either thereof.

20. The fusion protein of any one of claims 1 to 19, wherein the nanocage monomer peptide is modified to reduce an anti-nanocage monomer peptide immune response.

21. The fusion protein of claim 20, wherein the nanocage monomer peptide is at least partially or fully masked.

22. The fusion protein of claim 21, wherein the nanocage monomer peptide is at least partially glycan masked.

23. The fusion protein of claim 22, wherein the nanocage monomer peptide is fully glycan masked.

24. The fusion protein of any one of claims 20 to 23, wherein the nanocage monomer comprises at least one NXT and/or NXS glycosylation motif.

25. The fusion protein of claim 24, wherein the nanocage monomer comprises the amino acid sequence:
MLSKDIIKLLNEQVNKEMNSSNLYMSMSSWCYTHSLDGAGLFLFDHA
AEEYEHAKKLIVFLNENNVPVQLTSISAPEHKFEGLTQIFQKAYEHE
QHISESINNIVDHAIKSKDHATFNFLQWYVAEQHEEEVLFKDILDKI
ELIGNENHGLYLADQYVKGIAKSRKS
or a functional variant having at least 70% sequence identity thereto, or a functional fragment of either thereof, and wherein the sequence comprises a K77N and E79T mutation and/or an E99N and I101T mutations.

26. The fusion protein of any one of claims 1 to 25, wherein a plurality of the nanocage monomer peptides self-assemble into a nanocage.

27. The fusion protein of claim 26, wherein the immunogenic peptide decorates the interior and/or exterior surface of the nanocage.

28. The fusion protein of any one of claims 1 to 27, further comprising a peptide that provides exogenous T cell help and/or a peptide that provides autologous T cell help.

29. The fusion protein of claim 28, wherein the peptide that provides exogenous T cell help comprises a PADRE peptide and/or a peptide derived from a pathogenic molecule, such as a tetanus toxoid peptide.

30. The fusion protein of claim 29, wherein the PADRE peptide comprises the amino acid sequence AKFVAAWTLKAAA, or a functional variant thereof having at least 70% sequence identity thereto or a fragment of either thereof.

31. The fusion protein of any one of claims 28 to 30, wherein the peptide that provides autologous T cell help comprises a PfCSP T cell peptide epitope.

32. The fusion protein of any one of claims 28 to 31, wherein the peptide that provides exogenous T cell help and/or the peptide that provides autologous T cell help independently decorates the interior and/or exterior surface of the assembled nanocage.

33. The fusion protein of any one of claims 1 to 32, further comprising a linker between any one or more of the motifs, the nanocage monomer, and any further peptides, such as the peptide that provides exogenous T cell help and/or the peptide that provide autologous T cell help.

34. The fusion protein of claim 33, wherein the linker is a GGS linker.

35. The fusion protein of claim 34, wherein the linker comprises the amino acid sequence:
GGS;
GGGGSGGSGGSGGS;
and/or
GGGGGSGGSGGSGGS.

36. The fusion protein of any one of claims 1 to 35, comprising or consisting of the sequence:
KQPA-NPDP-NANPNVDP3-NANP5-Hpferr-PADRE;
KQPA-NPDP-NANPNVDP3-NANP18.5-Hpferr-PADRE;
KQPA-NPDP-NANPNVDP3-NANP5-LS-PADRE; and/or
KQPA-NPDP-NANPNVDP3-NANP18.5-LS-PADRE.

37. The fusion protein of any one of claims 1 to 36, comprising or consisting of the amino acid sequence:
MGILPSPGMPALLSLVSLLSVLLMGCVAETGWSHPQFEKLKENLYFQGKQPADGNPDPNAN
PNVDPNANPNVDPNANPNVDPNANPNANPNANPNANPNANPSRGGGGGSGGSGGSGGS
DIIKLLNEQVNKEMQSSNLYMSMSSWCYTHSLDGAGLFLFDHAAEEYEHAKKLIIFLNENNVP
VQLTSISAPEHKFEGLTQIFQKAYEHEQHISESINNIVDHAIKSKDHATFNFLQWYVAEQHEEE
VLFKDILDKIELIGNENHGLYLADQYVKGIAKSRKSGGSASAKFVAAWTLKAAA;
MGILPSPGMPALLSLVSLLSVLLMGCVAETGWSHPQFEKLKENLYFQGKQPADGNPDPNAN
PNVDPNANPNVDPNANPNVDPNANPNANPNANPNANPNANPNANPNANPNANPNANPNA
NPNANPNANPNANPNANPNANPNANPNANPNANPSRGGGGGSGGSGGSGGSDIIKLLNEQ
VNKEMQSSNLYMSMSSWCYTHSLDGAGLFLFDHAAEEYEHAKKLIIFLNENNVPVQLTSISA
PEHKFEGLTQIFQKAYEHEQHISESINNIVDHAIKSKDHATFNFLQWYVAEQHEEEVLFKDILD
KIELIGNENHGLYLADQYVKGIAKSRKSGGSASAKFVAAWTLKAAA;
MGILPSPGMPALLSLVSLLSVLLMGCVAETGWSHPQFEKLKENLYFQGKQPADGNPDPNAN
PNVDPNANPNVDPNANPNVDPNANPNANPNANPNANPNANPSRGGGGGSGGSGGSGGS
MQIYEGKLTAEGLRFGIVASRFNHALVDRLVEGAIDAIVRHGGREEDITLVRVPGSWEIPVAA
GELARKEDIDAVIAIGVLIRGATPHFDYIASEVSKGLADLSLELRKPITFGVITADTLEQAIERAG
TKHGNKGWEAALSAIEMANLFKSLRGGSASAKFVAAWTLKAAA;
MGILPSPGMPALLSLVSLLSVLLMGCVAETGWSHPQFEKLKENLYFQGKQPADGNPDPNAN
PNVDPNANPNVDPNANPNVDPNANPNANPNANPNANPNANPNANPNANPNANPNANPNA
NPNANPNANPNANPNANPNANPNANPNANPNANPSRGGGGGSGGSGGSGGSMQIYEGKL
TAEGLRFGIVASRFNHALVDRLVEGAIDAIVRHGGREEDITLVRVPGSWEIPVAAGELARKEDI
DAVIAIGVLIRGATPHFDYIASEVSKGLADLSLELRKPITFGVITADTLEQAIERAGTKHGNKGW
EAALSAIEMANLFKSLRGGSASAKFVAAWTLKAAA;
and/or
MGILPSPGMPALLSLVSLLSVLLMGCVAETGWSHPQFEKLKENLYFQGKQPADGNPDPNAN
PNVDPNANPNVDPNANPNVDPNANPNANPNANPNANPNANPNANPNANPNANPNANPNA
NPNANPNANPNANPNANPNANPNANPNANPNANPSRGGGGGSGGSGGSGGSDIIKLLNEQ
VNKEMQSSNLYMSMSSWCYTHSLDGAGLFLFDHAAEEYEHAKKLIIFLNENNVPVQLTSISA
PEHNFTGLTQIFQKAYEHEQHISNSTNNIVDHAIKSKDHATFNFLQWYVAEQHEEEVLFKDIL
DKIELIGNENHGLYLADQYVKGIAKSRKSGGSASAKFVAAWTLKAAA;
or functional variants or fragments thereof.

38. A nucleic acid molecule encoding the fusion protein of any one of claims 1 to 37.

39. A vector comprising the nucleic acid molecule of claim 38.

40. A host cell comprising the vector of claim 39 and producing the fusion protein of any one of claims 1 to 37.

41. A vaccine comprising the fusion protein of any one of claims 1 to 37.

42. The vaccine of claim 41, further comprising an adjuvant.

43. An antibody that binds to the fusion protein of any one of claims 1 to 37.

44. A highly efficacious pre-erythrocytic subunit malaria vaccine.

45. A C-terminal truncated PfCSP antigen.

46. A boostable malaria vaccine.

47. The vaccine of claim 46, wherein the vaccine is PfCSP-based.

48. An anti-malaria immunogenic peptide comprising KQPAₐ, wherein a is at least about 1.

49. A glycan-masked nanocage monomer peptide.

50. The peptide of claim 49, wherein the nanocage monomer is ferritin, apoferritin, encapsulin, SOR, lumazine synthase, pyruvate dehydrogenase, carboxysome, vault proteins, GroEL, heat shock protein, E2P, or MS2 coat protein, or fragments thereof, or variants thereof.

51. The peptide of claim 50, wherein the nanocage monomer is provided as two or more self-assembling subunits.

52. The peptide of any one of claims 49 to 51, wherein the nanocage monomer peptide is from Helicobacter pylori.

53. The peptide of any one of claims 49 to 52, wherein the nanocage monomer peptide is not human.

54. The peptide of any one of claims 49 to 53, wherein the peptide further comprises a bioactive moiety.

55. The peptide of claim 54, wherein the bioactive moiety comprises an antibody or fragment thereof, an antigen, a detectable moiety, a pharmaceutical agent, a diagnostic agent, or combinations thereof.

56. The peptide of claim 55, wherein the bioactive moiety comprises an antigen.

57. The peptide of any one of claims 49 to 56, where a plurality of the nanocage monomer peptides self-assemble into a nanocage.

58. The peptide of claim 57, wherein the bioactive moiety decorates the interior and/or exterior surface of the nanocage.

59. The peptide of any one of claims 49 to 58, wherein the nanocage monomer peptide is at least partially or fully masked.

60. The peptide of claim 59, wherein the nanocage monomer peptide is at least partially glycan masked.

61. The peptide of claim 59, wherein the nanocage monomer peptide is fully glycan masked.

62. The peptide of any one of claims 49 to 61, wherein the nanocage monomer comprises at least one NXT and/or NXS glycosylation motif.

63. The peptide of claim 62, wherein the nanocage monomer comprises the amino acid sequence:
MLSKDIIKLLNEQVNKEMNSSNLYMSMSSWCYTHSLDGAGLFLFDHA
AEEYEHAKKLIVFLNENNVPVQLTSISAPEHKFEGLTQIFQKAYEHE
QHISESINNIVDHAIKSKDHATFNFLQWYVAEQHEEEVLFKDILDKI
ELIGNENHGLYLADQYVKGIAKSRKS
or a functional variant having at least 70% sequence identity thereto, or a functional fragment of either thereof, and wherein the sequence comprises a K77N and E79T mutation and/or an E99N and I101T mutations.

64. A method of treating and/or preventing malaria, comprising administering the protein, peptide, nucleotide, vector, or cell of any one of claims 1 to 63.

65. Use of the protein, peptide, nucleotide, vector, or cell of any one of claims 1 to 63 for treating and/or preventing malaria.

66. The protein, peptide, nucleotide, vector, or cell of any one of claims 1 to 63 for use in treating and/or preventing malaria.

67. The method, use, or protein, peptide, nucleotide, vector, or cell of any one of claims 64 to 66, wherein the preventative and/or treatment effect is boostable.

68. The method, use, or protein, peptide, nucleotide, vector, or cell of any one of claims 64 to 66, wherein the preventative and/or treatment effect persists for at least about 6 months or more, such as about 9 months or more, about 12 months or more, about 1, about 2, about 3, about 4, about 5, about 6, about 7, about 8, about 9, or about 10 years or more.
